(19) United States
(12) Patent Application Publication
(10) Pub. No.: US 2022/0211782 A1
(43) Pub. Date: Jul. 7, 2022

(54) COMPOSITION FOR TREATMENT, ALLEVIATION OR PROPHYLAXIS OF ACNE

(71) Applicant: Lactobio A/S, Copenhagen Ø (DK)

(72) Inventors: Charlotte Vedel, Copenhagen O (DK); Katja Billekop Olsen, Copenhagen O (DK); Soren Kjaerulff, Copenhagen (DK)

(73) Assignees: Lactobio A/S, Copenhagen O (DK); Lactobio A/S, Copenhagen O (DK)

(21) Appl. No.: 17/612,816

(22) PCT Filed: May 20, 2020
(86) PCT No.: PCT/EP2020/064124

§ 371 Date: Nov. 19, 2021

(30) Foreign Application Priority Data
May 20, 2019 (DK) PA 2019 00601
Jan. 31, 2020 (DK) PA 2020 00124

(51) Int. Cl. A61K 35/747; A61K 35/744; A61K 35/741; A61P 17/10

(52) U.S. Cl. CPC A61K 35/747; A61P 17/10; A61K 35/741; A61K 35/744

(57) ABSTRACT

A composition comprises one or more of bacterial strains wherein the one or more of bacterial strains exhibit a stronger inhibition of pathogenic C. acnes strains compared to non-pathogenic C. acnes strains.

### TECHNICAL FIELD OF THE INVENTION

The present invention relates to a composition comprising bacterial strains. In particular the present invention relates to a novel topical dermatological composition comprising bacterial strains for treating; alleviating; suppressing; prophylaxis; and/or preventing growth of a pathogenic micro-organism.

### Background of the Invention

Acne vulgaris (acne) is currently one of the most common and well-established dermatology conditions, affecting around 90% of the world's population at some point in their lifetime. The disease is a chronic, inflammatory dermatological condition of the pilosebaceous gland of the skin with a multifactorial pathogenesis. It is usually initiated during puberty by hormonal changes and further exacerbated by genetic factors.

Cutibacterium acnes (formerly Probionibacterium acnes) is a Gram-positive, anaerobic bacterium found in sebaceous follicles that form part of the natural flora of the pilosebaceous unit; however, elevated levels of specific ribotypes of C. acnes are found in acne sufferers. Within the microenvironment formed in the context of acne, C. acnes thrive in the increased sebum production, using it as a nutrient for growth. C. acnes levels within the pore itself further contribute to the development of comedones.

C. acnes can simultaneously stimulate inflammation by metabolizing triglycerides and the release of free fatty acids. Inflammation is initiated via complement activation and the production of pro-inflammatory mediators, which diffuse through the follicle wall. It is wellknown that C. acnes activate toll-like receptor 2 (TLR2) on monocytes and neutrophils. Activation of TLR2 then leads to the production of multiple proinflammatory cytokines, notably interleukin 12 (IL-12) and interleukin 8 (IL-8) and TNFα.

With an increased pressure and the recruitment of inflammatory mediators, the microcomedone may rupture with extrusion of immunogenic keratin, sebum, and bacteria into the dermis. This stimulates a further exaggerated inflammatory response. The degree of inflammation and the corresponding cell types involved determine whether pustules, inflamed papules, nodules, or cysts develop on the skin. In severe cases of acne with excess inflammation and tissue damage, hyperpigmentation and scarring may result.

Various treatments exist for the treatment of acne. In general, acne treatments work by reducing oil production, speeding up skin cell turnover, fighting bacterial infection, reducing the inflammation or doing all four. These types of acne treatments include over-the-counter topical treatments, antibiotics, oral contraceptives and cosmetic procedures.

Acne lotions may dry up the oil, kill bacteria and promote sloughing of dead skin cells. Over-the-counter (OTC) lotions are generally mild and contain benzoyl peroxide, sulphur, resorcinol, salicylic acid or sulphur as their active ingredient. Antibiotics may cause side effects, such as an upset stomach, dizziness or skin discoloration. These drugs also increase the skin's sun sensitivity and may reduce the effectiveness of oral contraceptives. For deep cysts, antibiotics may not be enough. Isotretinoin (Amnesteem, Claravis, Sotret) is a powerful medication available for scarring cystic acne or acne that doesn't respond to other treatments. However, isotretinoin has many side effects, such as dry skin, depression, severe stomach pain, and muscle/joint/back pain, and can cause birth defects in babies whose mothers use isotretinoin. Chemical peels and microdermabrasion may be helpful in controlling acne. These cosmetic procedures, which have traditionally been used to lessen the appearance of fine lines, sun damage, and minor facial scars, are most effective when used in combination with other acne treatments. They may cause temporary, severe redness, scaling and blistering, and long-term discoloration of the skin.

WO 2017/220525 describes the beneficial effect of a composition comprising probiotic bacteria selected Lactobacillus strains against the development of common skin pathogens resulting in acne, in particular L. plantarum, L. pentosus and/or L. rhamnosus, are used. It is described that the composition provided in WO 2017/220525 comprises acids, such as lactic acid produced by the selected Lactobacillus strains which acts as an important antimicrobial factor. The disadvantage of this invention is that an unspecific inhibition of C. acnes (as well as the natural skin microbiota) is provided, thus, both pathogenic ribo-types of C. acnes (causing development of acne vulgaris); and non-pathogenic ribo-types of C. acnes.

In addition to the negative side-effects caused by the currently available treatments, there is no treatment available that is targeted to a modulation of the skin microbiome and targeted to the pathogenic ribo-types of C. acnes. Thus, there exists a need in the art for methods and treatments being specifically targeted to pathogenic C. acnes with limited antimicrobial effect on the natural skin microbiota and thus supporting the natural skin microbiota to fight acne.

### SUMMARY OF THE INVENTION

Thus, an object of the present invention relates to a composition comprising bacterial strains that solves the above problems with the prior art.

In particular the present invention relates to a novel composition, preferably a novel topical dermatological composition, comprising a bacterial strain for treating; alleviating; suppressing; prophylaxis; and/or preventing growth of a pathogenic micro-organism on the skin of a mammal, such as a human which solves the above-mentioned problems of the prior art with acne skin infections, acne skin diseases, skin inflammation, drying of the skin and scars. Caused by acne infections.

Thus, one aspect of the invention relates to a composition comprising one or more of bacterial strains wherein the one or more of bacterial strains exhibit a stronger inhibition of pathogenic C. acnes strains compared to non-pathogenic C. acnes strains.

A further aspect of the present invention relates to a composition comprising one or more of bacterial strains capable of co-aggregating stronger to one or more pathogenic C. acnes strains relative to the capability of the one or more bacterial strains to co-aggregation to one or more non-pathogenic C. acnes strains.

Another aspect of the present invention relates to a product comprising the composition according to the present invention for use as a medicament.

Yet an embodiment of the present invention relates to a product comprising the composition according to the present invention for use in treating; alleviating, suppressing; prophylaxis; and/or preventing growth of a pathogenic micro-organism.

A further embodiment of the present invention relates to a product comprising the composition according to the present invention comprising one or more bacterial species capable of co-aggregating one or more C. acnes strains for use in treating; alleviating, suppressing; prophylaxis; and/or preventing of acne vulgaris.

An even further aspect of the invention relates to a composition comprising one or more lactic acid bacterial species capable of co-aggregating one or more C. acnes strains, wherein the one or more lactic acid bacterial species may be selected from a bacterial strain having a genetic homology of at least 95% to one or more of the bacterial strains selected from the group consisting of:
LB356R (Lactobacillus plantarum LB356R, deposited under DSM 33094)
LB244R (Lactobacillus plantarum LB244R, deposited under DSM 32996)
LB349R (Leuconostoc mesenteriodes LB349R, deposited under DSM 33093)
LB10G (Weissella viridescens LB10G, deposited under DSM 32906)

An even further aspect of the invention relates to the use of a composition comprising one or more bacterial strains for reducing or avoiding attachment of one or more C. acnes strains to a surface and/or for reducing or avoiding biofilm formation of one or more C. acnes strains on a surface.

### DETAILED DESCRIPTION OF THE INVENTION

Prior to discussing the present invention in further details, the following terms and conventions will first be defined:

The term “ribotype” refers to specific strains of C. acnes. The ribotyped strains were characterized as in Fitz-Gibbon et al. J. Investigative Dermatology 133:2152-60 (2013).

The term “phylotype” refers to specific strains of C. acnes. The phylotyped strains were characterized as in McDowell et al. PLoS ONE 8(9): e70897 (2013).

The term “pathogenic C. acnes” refers to the ripotypes and phylotypes being associated with acne disease and acne infected skin.

A “therapeutical effective amount” of a compound with respect to the subject method of treatment refers to an amount of the compounds) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit risk ratio applicable to any medical treatment.

As used herein, the term ‘treating” or ‘treatment” includes reversing, reducing, or arresting the symptoms, clinical signs, and underlying pathology of a condition in a manner to improve or stabilize a subject's condition.

Accordingly, C. acnes is a major commensal of the human skin. It contributes to maintaining healthy skin by inhibiting the invasion of common pathogens, such as Staphylococcus aureus and Streptococcus pyogenes. It does so by hydrolyzing triglycerides and releasing free fatty acid that contributes to the acidic pH of the skin surface. On the other hand, C. acnes has been historically linked to acne vulgaris, a chronic inflammatory disease of the pilosebaceous unit. A metagenomic study demonstrated that C. acnes was a dominant bacterium in the pilosebaceous unit in both healthy individuals and acne patients.

It has found that at strain level, the population structures of C. acnes were different between the two groups of strains. These findings suggested that microbe-related human diseases are often caused by certain strains of a species rather than the entire species, in line with the studies of other diseases. C. acnes has been classified into three distinct types, first revealed two distinct phenotypes of C. acnes, known as types I and II, that could be distinguished based on serological agglutination tests and cell wall sugar analysis. C. acnes types I and II was distinguished by monoclonal antibody typing. Furthermore, their phylogenetic analysis of C. acnes strains based on the nucleotide sequences of the recA gene and a more variable hemolysin/cytotoxin gene (tly) demonstrated that types I and II represent distinct lineages. Strains within the type I lineage could be further split into two clades, known as types IA and IB, both associated with acne disease. An additional phylogenetic group of C. acnes, known as type III was described later. Recent studies based on multilocus sequence typing (MLST) further sub-divided C. acnes into closely related clusters, some of which were associated with various diseases including acne. Results from the Human Microbiome project (HMP) generated knowledge about the specific ribotypes of C. acnes strains being associated with health and disease (McDowell et al. 2013, PLoS ONE 8(9): e70897, Dreno et al. 2018, JEADV 32 (suppl. 2), 5-14. doi:10.1111/jdv.15043). Specific ribotypes of C. acnes are known to be non-pathogenic and part of the natural healthy skin microbiota, other specific ribotypes of C. acnes are identified as being pathogenic and dominant in acne infections. Thus, it has been demonstrated that RT4 and RT5 is strongly associated with acne and RT6 is associated with healthy skin. Other C. acnes strains including ribotypes RT7, RT8, RT9 and RT10 may also contribute to development of the disease (Fitz-Gibbon et al. 2013, J. Invest. Dermatol. 133:2152-60. doi:10.1038/jid.2013.21).

Skin with acne does not harbour more C. acnes than healthy skin, it is an un-balance between the pathogenic C. acnes ribotypes and the non-pathogenic ribotypes.

Hence, an improved antimicrobial activity against pathogenic C. acnes would be advantageous.

The present invention relates to probiotic bacteria and a composition comprising the probiotic bacteria for use in the prevention or treatment of infections from pathogenic microorganisms, such as Cutibacterium acnes infections.

The inventors of the present invention surprisingly found that some bacterial strains may inhibit the growth of pathogenic ribotypes of C. acnes, whereas the growth of non-pathogenic ribotypes of C. acnes. Are significantly less affected, or substantially not affected at all.

In an embodiment of the present invention the one or more C. acnes strains may preferably be one or more pathogenic C. acnes strains.

The one or more C. acnes strains, or the one or more pathogenic C. acnes strains, may be selected from a Cutibacterium acnes ribotype RT4; ribotype RT5; ribotype RT7; ribotype RT8; ribotype RT9; and/or ribotype RT10. Preferably, the one or more pathogenic C. acnes strains, may be selected from a Cutibacterium acnes ribotype RT4; ribotype RT5; and/or ribotype RT8

In an embodiment of the present invention the one or more C. acnes strains may be selected from the group consisting of Cutibacterium acnes HL110PA1; Cutibacterium acnes HL053PA2; Cutibacterium acnes HL043PA1; and/or Cutibacterium acnes HL038PA1.

The present invention provides new microbial strains and new composition which may inhibit growth of pathogenic C. acnes without contributing to skin irritation and dryness.

In the context of the present invention the terms “inhibit” and “inhibition” may relate to full or partial inhibition.

Thus, a preferred embodiment of the present invention relates to a composition comprising one or more of bacterial strains capable of co-aggregating one or more C. acnes strains.

Preferably, a preferred embodiment of the present invention relates to a composition comprising one or more of bacterial strains wherein the one or more of bacterial strains exhibit a stronger inhibition of pathogenic C. acnes strains compared to non-pathogenic C. acnes strains.

The inventors surprisingly found that specific bacterial strains, in particular specific lactic acid bacterial strains, shows to act stronger towards pathogenic C. acnes strains than non-pathogenic C. acnes strains. This finding allows to provide a composition comprising said specific bacterial strains suitable for the treatment, alleviating, suppressing; prophylaxis of one or more pathogenic C. acnes strains,

Preferably, inhibition relates to a growth inhibition of a C. acnes strains, preferably a growth inhibition of pathogenic C. acnes strain; co-aggregation inhibition of a C. acnes strain, preferably co-aggregation inhibition of pathogenic C. acnes strains; and/or biofilm inhibition of a C. acnes strain, preferably, biofilm inhibition of pathogenic C. acnes strains. Most preferably, inhibition relates to inhibition of co-aggregation of a C. acnes strain, preferably inhibition of co-aggregation of pathogenic C. acnes strains; and/or biofilm inhibition of a C. acnes strain, preferably, biofilm inhibition of pathogenic C. acnes strains

In an embodiment of the present invention the composition comprising one or more of bacterial strains may be capable of co-aggregating stronger to one or more pathogenic C. acnes strains relative to the capability of the one or more bacterial strains to co-aggregation to one or more non-pathogenic C. acnes strains.

The one or more bacterial strains may preferably be one or more lactic acid bacterial strains.

The one or more bacterial strains (preferably the one or more lactic acid bacterial strains) is capable of co-aggregate with strong aggregation intensity with one or more pathogenic C. acnes strains. By visual inspection of the aggregation, the aggregation size may be determined as an indication of the aggregation intensity with the one or more pathogenic C. acnes strains.

In a preferred embodiment of the present invention the one or more bacterial strains (preferably the one or more lactic acid bacterial strains) is capable of co-aggregate with weak aggregation intensity with one or more non-pathogenic C. acnes strains.

In an even preferred embodiment of the present invention the composition and/or the one or more bacterial strains (preferably the one or more lactic acid bacterial strains) is capable of co-aggregate with a stronger aggregation intensity with one or more pathogenic C. acnes strains, relative to the aggregation intensity with one or more non-pathogenic C. acnes strains

The formation of co-aggregation may be scored visually from 1-5 (the higher the value the stronger the aggregation intensity) using the following scale:
1: No aggregation
2: Visual initial aggregation
3: Formation of aggregates <0.5 mm
4: Formation of aggregates >0.5 mm and <1 mm
5: Formation of aggregates >1 mm

In an embodiment of the present invention the inhibition of pathogenic C. acnes strains is 10% stronger or more relative to the inhibition of the non-pathogenic C. acnes strains, such as 15% stronger or more, e.g. 20% stronger or more, such as 30% stronger or more, e.g. 40% stronger or more, such as 50% stronger or more, e.g. 75% stronger or more, such as 100% stronger or more, e.g. 150% stronger or more, such as stronger 200% or more, e.g. stronger 300% or more.

In another embodiment of the present invention the inhibition of non-pathogenic C. acnes strains is 90% or less of the inhibition of the pathogenic C. acnes strains, such as 80% or less, e.g. 70% or less, such as 60% or less, e.g. 50% or less, such as 40% or less, e.g. 30% or less, such as 20% or less.

In the context of the present invention the terms “stronger” or “stronger intensity” are used interchangeable and relates to an increased effect on the non-pathogenic C. acnes strains relative to the non-pathogenic C. acnes strains. E.g. in respect of co-aggregation where a stronger effect may be resulting in formation of larger aggregates comprising pathogenic C. acnes strains compared to the size of the aggregates formed comprising non-pathogenic C. acnes strains. In respect of the formation of biofilm the stronger effect may be resulting in formation of a smaller biofilm with reduced number of pathogenic C. acnes strains and/or formation of a biofilm with higher ratio of non-pathogenic C. acnes strains relative to the pathogenic C. acnes strains. In respect of the increased effect on the growth the stronger effect may be resulting in an increased growth inhibition of the pathogenic C. acnes strains relative to the growth of non-pathogenic C. acnes strains.

The inventors of the present invention surprisingly found that by improving co-aggregation of C. acnes strains provided by the present invention, biofilm formation on a surface may be limited, reduced or prevented. The formation of a biofilm begins with the attachment of free-floating microorganisms to a surface. If these first colonists are not prevented from attaching to the surface, they can anchor themselves more permanently to the surface and attract other microorganisms to attach and the community may be growing and preparing a biofilm.

The composition according to the present invention, surprisingly showed to limit, reduce or prevent:
1) the initial first colonists of C. acnes strains to attach to a surface; and/or
2) preventing C. acnes strains to attach to colonized microorganisms.

It is believed that one of the abilities of C. acnes to cause diseases or infections may be related to the attachment to surfaces of human cells and initiation of C. acnes biofilm. Such biofilm formation of C. acnes makes the infection much more difficult to prevent or treat as the biofilm formation protect the C. acnes bacteria from antimicrobial activity.

Biofilm formation of pathogenic C. acnes may provide an intrinsic resistance to the C. acnes strain which is the innate ability of a bacterial species to resist activity of a particular antimicrobial agent through its inherent structural or functional characteristics, which allow tolerance of a particular drug or antimicrobial compound.

The inventors of the present invention surprisingly found that the bacterial strains according to the present invention, e.g. the lactic acid bacterial strains of the present invention, were able to prevent C. acnes from initial binding to a surface and inhibition of the following creation of biofilm.

In an embodiment of the present invention the bacterial strains according to the present invention, e.g. the lactic acid bacterial strains of the present invention, may be able to prevent C. acnes from initial binding to a surface and inhibition of the following creation of biofilm.

Preferably, the surface may be selected from a cell, from the mammalian body, such as the human body, wherein the body may be the skin or the mucous membranes of the mammal, such as a human.

A preferred embodiment of the present invention relates to a composition comprising one or more bacterial strains capable of inhibiting, reduce, limit and/or prevent attachment to a surface of one or more C. acnes strains and/or inhibiting, reduce, limit and/or prevent biofilm formation on a surface of one or more C. acnes strains.

Preferably, the inhibition, reduction, limitation and/or prevention of one or more C. acnes strains to attach and/or or forming biofilm on a surface may be one or more pathogenic C. acnes strains.

Attachment and/or biofilm formation of one or more C. acnes strains, preferably, one or more pathogenic C. acnes strains, to a surface may be reduced by 15% (w/w) or more relative to samples where no bacterial strains has been added, such as by 25% (w/w) or more, e.g. by 50% (w/w) or more, such as by 75% (w/w) or more, e.g. by 90% (w/w) or more, such as by 95% (w/w) or more, e.g. by 98% (w/w) or more.

In an embodiment of the present invention the one or more bacterial strains (preferably the one or more lactic acid bacterial strains) is capable of co-aggregate with strong aggregation intensity after 1 hour and/or after 24 hours of 4 to 5 (on a scale from 1-5). Preferably, the one or more bacterial strains (preferably the one or more lactic acid bacterial strains) is capable of co-aggregate with strong aggregation intensity after 1 hour. The strong aggregation intensity may be by a score of 4 to 5 (on a scale from 1-5).

The faster a strong co-aggregation is formed the better and more specific, e.g. one or more pathogenic C. acnes strains, may be.

On the other hand, the one or more bacterial strains, preferably the one or more lactic acid bacterial strains, may for co-aggregates with one or more non-pathogenic C. acnes strains. Such co-aggregation with one or more non-pathogenic C. acnes strains may be considered having a weak aggregation intensity.

In an embodiment of the present invention the one or more bacterial strains (preferably the one or more lactic acid bacterial strains) is capable of co-aggregate with weak aggregation intensity, a value 1-3 (on a scale from 1-5) after 1 hour and/or after 24 hours.

Since there is a significant difference in the co-aggregation of the one or more bacterial strains according to the present invention with pathogenic C. acnes strains and with non-pathogenic C. acnes strains, where the strongest co-aggregation is found between the one or more bacterial strains and the pathogenic C. acnes strains, the inventors of the present invention surprisingly found that it is possible to treat; alleviate, suppress; prophylaxis; and/or preventing development acne vulgaris in a mammal, caused by pathogenic C. acnes strains. At the same time the inventors surprisingly found that it may be possible at the same time to maintain the non-pathogenic C. acnes strains (or part hereof) as part of the healthy skin microbiota.

In an embodiment of the present invention the one or more lactic acid bacterial strains may be a Lactobacillus strain; a Leuconostoc strain; and/or a Weissella strain. Preferably, the bacterial strains may be a Lactobacillus strain.

Preferably the Lactobacillus strains may be selected from Lactobacillus planetarium. In an embodiment of the present invention the Lactobacillus planetarium may be selected from Lactobacillus planetarium LB356R, deposited under DSM 33094 or from Lactobacillus planetarium LB244R, deposited under DSM 32996.

Preferably, the Leuconostoc strains may be selected from Leuconostoc mesenteriodes. In an embodiment of the present invention the the Leuconostoc mesenteriodes may be selected from Leuconostoc mesenteriodes LB349A, deposited under DSM 33093.

Preferably, the Weissella strains may be selected from Weissella viridescens. In an embodiment of the present invention the Weissella viridescens may be selected from Weissella viridescens LB10G, deposited under DSM 32906.

In another embodiment of the present invention the one or more lactic acid bacterial strains capable of co-aggregating one or more C. acnes strains may be a combination of:
a Lactobacillus planetarium, preferably selected from Lactobacillus planetarium LB356R, deposited under DSM 33094 and/or from Lactobacillus planetarium LB244R, deposited under DSM 32996;
a Leuconostoc mesenteriodes, preferably, selected from Leuconostoc mesenteriodes LB349A, deposited under DSM 33093; and/or
a Weissella viridescens, preferably, selected from Weissella viridescens LB10G, deposited under DSM 32906.

Preferably, the one or more lactic acid bacterial species capable of co-aggregating one or more C. acnes strains may be selected from a bacterial strain having a genetic homology of at least 95% to one or more of the bacterial strains selected from the group consisting of:
LB356R (Lactobacillus plantarum LB356R, deposited under DSM 33094)
LB244R (Lactobacillus plantarum LB244R, deposited under DSM 32996)
LB349R (Leuconostoc mesenteriodes LB349R, deposited under DSM 33093)
LB10G (Weissella viridescens LB10G, deposited under DSM 32906)

A preferred embodiment of the present invention relates to a composition comprising one or more lactic acid bacterial species capable of co-aggregating one or more C. acnes 35 strains, the one or more lactic acid bacterial species may be selected from a bacterial strain having a genetic homology of at least 95% to one or more of the bacterial strains selected from the group consisting of:
LB356R (Lactobacillus plantarum LB356R, deposited under DSM 33094)
LB244R (Lactobacillus plantarum LB244R, deposited under DSM 32996)
LB349R (Leuconostoc mesenteriodes LB349R, deposited under DSM 33093)
LB10G (Weissella viridescens LB10G, deposited under DSM 32906)

In another embodiment of the present invention the one or more lactic acid bacterial species capable of co-aggregating one or more C. acnes strains may be selected from a bacterial strain having a genetic homology of 96%; such as at least 97%; e.g. at least 98%; such as at least 99%; e.g. at least 99.5%; such as at least 99.8%; e.g. at least 99.9%; such as 100% (identical) to one of the bacterial strains selected from the group consisting of:
LB356R (Lactobacillus plantarum LB356R, deposited under DSM 33094)
LB244R (Lactobacillus plantarum LB244R, deposited under DSM 32996)
LB349R (Leuconostoc mesenteriodes LB349R, deposited under DSM 33093)
LB10G (Weissella viridescens LB10G, deposited under DSM 32906)

In yet an embodiment of the present invention the one or more lactic acid bacterial species capable of co-aggregating one or more C. acnes strains may be selected from a bacterial strain selected from the group consisting of:
LB356R (Lactobacillus plantarum LB356R, deposited under DSM 33094)
LB244R (Lactobacillus plantarum LB244R, deposited under DSM 32996)
LB349R (Leuconostoc mesenteriodes LB349R, deposited under DSM 33093)
LB10G (Weissella viridescens LB10G, deposited under DSM 32906).

In the present context the term “genetic homology” relates to a deviation in the genetic sequence of the bacterial strain relative to the deposited bacterial strains.

The effect of the bacterial strain (and/or the composition) according to the present invention on the pathogenic microorganism may be significant.

The composition according to the present invention may be capable of inhibiting growth of Cutibacterium acnes. In an embodiment of the present invention the growth of pathogenic C. acnes may be reduced by at least 20%; such as reduced by at least 30%, e.g. reduced by at least 40%; such as reduced by at least 50%, e.g. reduced by at least 60%.

In a preferred embodiment the bacterial strain according to the present invention may be an isolated bacterial strain.
The present invention discloses microorganisms which are associated through the functional relationship with one another to form a uniform idea according to the invention, such that they share the properties and/or effects, namely that they inhibit growth of pathogenic microorganisms, like C. acnes, and/or reduce the colonization level of pathogenic microorganisms, like C. acnes, associated with skin diseases. These lactic acid bacteria include in particular microorganisms or analogs, fragments, lysates, derivatives, mutants or combinations thereof selected from the group comprising the following new isolated microorganisms deposited with the German Collection for Microorganisms and Cell Cultures:
LB356R (Lactobacillus plantarum LB356R, deposited under DSM 33094)
LB244R (Lactobacillus plantarum LB244R, deposited under DSM 32996)
LB349R (Leuconostoc mesenteriodes LB349R, deposited under DSM 33093)
LB10G (Weissella viridescens LB10G, deposited under DSM 32906).

The present invention encompasses a composition comprising at least one of these new lactic acid bacteria and a composition comprising any combination of these strains and analogs, fragments, lysates, derivatives, mutants hereof.

Acne infections can be co-infected with Staphylococcus e.g. Staphylococcus aureus which can worsen the disease.

In an embodiment of the present invention the composition according to the present invention may also comprise antimicrobial activity. In particular the invention relates to a composition comprising one or more bacterial strains and having antimicrobial activity against Staphylococcus being associated with acnes co-infections.

In the context of the present invention the term “bacterial strains” relates to bacterial strains according to the present invention. Preferably, bacterial strains relate to lactic acid bacterial strains according to the present invention.

In the context of the present invention the term “co-aggregation” relates to the aggregation of pathogenic bacteria, such as pathogenic C. acnes strains along with another bacteria, namely the one or more bacterial strain, e.g. the lactic acid bacterial strain, according to the present invention.

Preferably the co-aggregate may be clearly visible by the human eye.

In an embodiment of the present invention the co-aggregation forms aggregates within 24 hours having an aggregate particle size above 0.1 mm; such as above 0.25 mm; e.g. above 0.5 mm; such as above 0.75 mm; e.g. above 1.0 mm; such as in the range of 0.1-0.5 mm; preferably, in the range of 0.5-1.0 mm; even more preferably above 1.0 mm.

In another embodiment of the present invention the co-aggregation forms aggregates within 1 hour having an aggregate particle size above 0.1 mm; such as above 0.25 mm; e.g. above 0.5 mm; such as above 0.75 mm; e.g. above 1.0 mm; such as in the range of 0.1-0.5 mm; preferably, in the range of 0.5-1.0 mm; even more preferably above 1.0 mm.

Yet another aspect of the present invention relates to a composition according to the present invention for use in treating; alleviating, suppressing; prophylaxis; and/or preventing growth of a pathogenic micro-organism.

The inventors of the present invention provides a therapeutic composition for the treatment or prevention of an infection, comprising a therapeutically-effective concentration of one or more bacterial strains within a pharmaceutically-acceptable carrier suitable for administration to the gastrointestinal tract of a mammal and/or a topical administration on the skin or mucous membranes of a mammal, wherein said of one or more bacterial strains possesses the ability to inhibit growth of pathogens, colonization rate and initial attachment of the pathogen to the infection site.

In an embodiment of the present invention the composition may be a topical composition (for topical treatment) or an oral composition (for oral treatment) or a rectal composition (for rectal treatment). Preferably, the composition may be a topical composition, preferably a topical composition for the topical treatment of acne vulgaris.

The treatment of acne according to the present invention may be a combined treatment involving a topical treatment and an oral treatment.

In a further embodiment of the present invention the composition may be a topical dermatological composition.

A preferred embodiment of the present invention relates to a composition comprising one or more of the bacterial strains according to the present invention.

The concentration of the bacterial strain, preferably the one or more viable strain, may be in the range of 10³ to 10¹⁴ colony forming units (CFU); such as in the range of 10⁵-10¹³ CFU; e.g. in the range of 10⁶-10¹² CFU; such as in the range of 10⁷-10¹¹ CFU; e.g. in the range of 10⁸-10¹⁰ CFU; such as about 10⁹ CFU.

The concentration of the bacterial strain, preferably one or more dead/inactivated strains, one or more strain lysate; one or more strain metabolites may be in a concentration range of 0.001% (w/w) to 20% (w/w) relative to the topical composition; such as in the range of 0.01% (w/w) to 18% (w/w); e.g. in the range of 0.1% (w/w) to 15% (w/w); such as in the range of 0.5% (w/w) to 10% (w/w); e.g. in the range of 1% (w/w) to 5% (w/w).

The bacterial strains defined herein may be provided in the composition according to the present invention in the form of a dead bacterial strain. The dead bacterial strain may be provided as whole dead cells or as lysates, metabolites, derivatives, analogs, fractions or extracts obtained from the dead cell.

In an embodiment of the present invention the one or more bacterial strains may be provided as one or more viable strains, one or more dead or inactivated strains, one or more strain lysate; one or more strain metabolites or a combination hereof.

Preferably, the strain lysate may be provided by acid treatment, e.g. using HCl, and/or by sonication.

In the composition the bacterial strain according to the present invention may be provided as one or more viable strains, one or more dead or inactivated strains, one or more strain lysate; one or more strain metabolites; one or more analogue, one or more fragment, one or more derivative, one or more mutant or combination thereof, where the lysate; one or more strain metabolites; one or more analogue, one or more fragment, one or more derivative, one or more mutant or combination thereof (as obtained from the bacterial strains according to the present invention) may treat; alleviate; suppress; prophylaxis; and/or prevent growth of at least one pathogenic microorganism, e.g. C. acnes.

C. acnes is a normal habitant of the human gut microbiome and colonize the intestinal gut. Thus, gut colonization of the pathogenic ribotypes may also influence skin microbiota and therefore the probiotic bacteria with antimicrobial activity against C. acnes may advantageously be used as an oral composition.

In an embodiment of the present invention wherein the composition may be a topical composition and/or an oral composition, preferably the composition is a topical composition.

In an embodiment of the present invention wherein the composition may be a topical composition and/or an oral composition, preferably the composition is an oral composition.

In an embodiment of the present invention the composition may have an acidic pH-value, preferably pH-value of the composition is pH 6.5 or below; such as pH 6 or below; e.g. pH 5.5 or below; such as pH 5 or below; e.g. pH 4.5 or below; such as in the range of pH 4-6.5; e.g. in the range of pH 4.5-6.0; such as in the range of pH 4.7-5.5.

In yet an embodiment of the present invention the composition may, when applied to a surface, such as the skin of a human, result in an acidic pH-value of the surface (the skin), preferably pH-value is pH 6.5 or below; such as pH 6 or below; e.g. pH 5.5 or below; such as pH 5 or below; e.g. pH 4.5 or below; such as in the range of pH 4-6.5; e.g. in the range of pH 4.5-6.0; such as in the range of pH 4.7-5.5.

The composition according to the present invention may preferably comprise a pharmaceutically or cosmetically acceptable vehicle or excipient. In an embodiment of the present invention the composition may be provided in solid form, liquid form, viscous form, emulsion or as a dried form.

The composition for oral consumption may preferably be formulated into a paste, a soft gelatin capsule, a hard gelatin capsule, a powder, a talc, a granule, a bead, a pastille, an effervescent tablet, lozenges, buccal tablets, chewable tablets, sublingual tablets, an oil, a liquid, a solution, a tincture, an emulsion, a juice, a concentrate, a syrup, a spray, a mist, a drinking ampoule, a gel, a gum, a tablet, a coated pill or as a food or a feed product or a drink.

The composition for topical application may preferably be formulated into a paste; a talc; a powder; a lotion; a custard; a foam; a cream; an oil, a serum or an ointment.

In an embodiment of the present invention the topical composition may be a powder composition comprising hydrated magnesium silicate (talc) and at least one of the bacterial strains of the invention.

In further embodiment according to the present invention the powder composition for topical application comprises hydrated magnesium silicate, at least one carbohydrate, and at least one of the bacterial strains of the invention.

In a preferred embodiment the topical composition may be formulated into a lotion; a custard; a foam; a crème; an ointment, an oil, a serum, a gel or an emulsion.

In a preferred embodiment the topical composition is a vegetable oil.

In a preferred embodiment the treatment is a combined treatment of both a topical composition and an oral composition comprising the bacterial strains according to the present invention.

The composition may, in addition to the bacterial strains according to the present invention, further comprise other probiotics, prebiotics, antimicrobials, antibiotics or other active antibacterial substances and/or may preferably also contain one or more of the following substances selected from antioxidants, vitamins, coenzymes, fatty acids, amino acids and cofactors.

In another embodiment of the present invention, the bacterial strains according to the present invention may be combined with:
a therapeutically-effective dose of an antibiotic. Either as a co-treatment or following an antibiotic therapy;
a therapeutic concentration of antibiotic including, but not limited to: Fusidic acid; Vancomycin; Gentamicin; Oxacillin; Tetracyclines; Nitroflurantoin; Chloramphenicol; Clindamycin; Trimethoprim-sulfamethoxasole; a member of the Cephlosporin antibiotic family (e.g., Cefaclor, Cefadroxil, Cefixime, Cefprozil, Ceftriaxone, Cefuroxime, Cephalexin, Loracarbef, and the like); a member of the Penicillin family of antibiotics (e.g., Ampicillin, Amoxicillin/Clavulanate, Bacampicillin, Cloxicillin, Penicillin VK, and the like); with a member of the Fluoroquinolone family of antibiotics (e.g., Ciprofloxacin, Grepafloxacin, Levofloxacin, Lomefloxacin, Norfloxacin, Ofloxacin, Sparfloxacin, Trovafloxacin, and the like); or a member of the Macrolide antibiotic family (e.g., Azithromycin, Erythromycin, and the like);
a therapeutically-effective dose of an anti-inflammatory drug. Either as a co-treatment or following a therapy; and/or
a therapeutic concentration of anti-inflammatory drug.

In an embodiment of the present invention the composition may be a pharmaceutical, veterinary or food product or a food supplement or a food supplement composition. The composition (preferably for oral administration) may preferably contains one or more thickeners, and/or one or more sweeteners and/or one or more artificial sweeteners, wherein the thickener is preferably selected from cellulose ether, polysaccharides, selected from the group comprising xanthan gum, gelatin, highly dispersed silicon dioxide, starch, carragenans, alginates, tragacanth, agar, gum arabic, pectin and polyvinyl esters, and the sweetener is selected from the group comprising glucose, fructose, sucrose, glucose syrup, sorbitol, mannitol, xylitol, maltitol, stewia, saccharine, sodium cyclamate, acesulfame K and/or aspartame.

Preferred foods and nutritional supplements in the sense of the invention may comprise effervescent tablets, vitamin tablets, dietary supplements, mineral tablets, trace element tablets, beverage powders, beverages, juices, milk beverages, yogurts, mineral water, uncarbonated water, bonbons, chewable tablets, juice or syrup, coated pills and pastilles as well as aerosols.

Furthermore, the composition may also contain builders, enzymes, electrolytes, pH regulators, thickeners, prebiotics, optical brighteners, graying inhibits, foam regulators and/or coloring agents.

Nowhere in the prior art has there been disclosed the use of probiotic strains to prevent or treat infections of specific pathogenic acne infection caused by the pathogenic ribotypes of C. acnes.

It was completely surprising that a group of lactic acid bacteria could be identified that had identical advantageous properties. No bacteria, in particular no lactic acid bacteria combine these properties of treating; alleviating, suppressing; prophylaxis; and/or preventing growth of a pathogenic micro-organism while also being non-pathogenic and not causing any damage to or influence on the skin or to the microbiota.

It will be understood that in the following, preferred embodiments referred to in relation to one broad aspect of the invention are equally applicable to each of the other broad aspects of the present invention described above. It will be further understood that, unless the context dictates otherwise, the preferred embodiments described below may be combined. When used herein, the term topical includes references to formulations that are adapted for application to body surfaces (e.g. the skin or mucous membranes).

An embodiment of the present invention relates to a composition according to the present invention for use in treating; alleviating, suppressing; prophylaxis; and/or preventing growth of a pathogenic micro-organism.

An embodiment of the present invention relates to a composition according to the present invention for use in healing the scars and wounds caused by bacterial infection.

More preferably the present invention may provide a composition a defined herein for use in the treatment, alleviating, suppressing; prophylaxis of one or more pathogenic bacterial infection in a mammal.

More preferably the present invention may provide a composition a defined herein for use in the preventing growth of a pathogenic micro-organism.

The bacterial infection may preferably be a C. acnes infection in a mammal.

An embodiment of the present invention relates to a composition as defined herein for use in the treatment; alleviation; suppression; and/or the prophylaxis of diseases resulting from C. acnes infections

In a further embodiment of the present invention the composition comprising at least one of the bacterial strains (or lysates, metabolites, derivatives, analogs, fractions or extracts thereof) to treat skin infections caused by C. acnes.

An embodiment of the preferred invention relates to a composition for use as a prophylaxis or medical treatment of C. acnes infections.

The microorganisms may advantageously be present in viable or killed/dead form in the composition. The bacterial strain may be provided in an encapsulated, micro-encapsulated, spray-dried and/or lyophilized form. Furthermore, the bacterial strain may be provided in the form of a cell lysate, metabolites, derivatives, analogs, fractions or extracts.

The microorganisms may advantageously be present in viable form in the composition. The bacterial strain may be provided in an encapsulated, micro-encapsulated, spray-dried and/or lyophilized form in an oil formulation.

In a preferred embodiment of the invention the oil is a vegetable selected from almond oil, hemp oil, CBD oil, cannabis oil, Evening prim rose, Borage oil, Almond sweet oil, Rose Hip oil, Jojoba Golden oil, Jojoba oil, Chamomile oil, Calendula oil, Sea buck-thorn oil, Jafflower oil and sesame oil.

In a preferred embodiment of the invention composition comprises at least one oil selected from almond oil, hemp oil, CBD oil, cannabis oil, Evening prim rose, Borage oil, Almond sweet oil, Rose Hip oil, Jojoba Golden oil, Jojoba oil, Chamomile oil, Calendula oil, Sea buck-thorn oil, Jafflower oil and/or sesame oil.

In a preferred embodiment of the invention composition comprises at least 10% (w/w) Jojoba oil or Jojoba wax or Jojoba golden oil.

In an embodiment of the present invention the bacterial strain may be present in the composition in an amount by weight of 0.001% (w/w) to 20% (w/w) relative to the topical composition; such as in the range of 0.01% (w/w) to 18% (w/w); e.g. in the range of 0.1% (w/w) to 15% (w/w); such as in the range of 0.5% (w/w) to 10% (w/w); e.g. in the range of 1% (w/w) to 5% (w/w).

A preferred embodiment of the present invention involves the administration of from approximately 1×10³ to 1×10¹⁴ CFU of viable bacteria per day, more preferably from approximately 1×10⁴ to 1×10¹⁰, and most preferably from approximately 5×10⁴ to 1×10⁹ CFU of viable bacteria per day. Where the condition to be treated involves antibiotic resistant pathogens and the patient is an adult, the typical dosage is approximately 1×10² to 1×10¹⁴ CFU of viable bacteria per day, preferably from approximately 1×10⁸ to 1×10¹⁰, and more preferably from approximately 2.5×10⁸ to 1×10¹⁰ CFU of viable bacteria per day.

The present invention relates to novel bacterial strains and the general reference in the claims relates to viable cells, dead/killed cells and lysates, metabolites, derivatives, analogs, fractions or extracts thereof as well as compositions comprising such viable cells, dead/killed cells and lysates, metabolites, derivatives, analogues, fractions or extracts thereof.

The composition according to the present invention may be suitable for the treating; alleviating, suppressing; prophylaxis; and/or preventing growth of a pathogenic micro-organism, such as C. acnes in children, teenagers, healthy persons, women, the elderly, immunosuppressed people, people with single-occurrence or recurring C. acnes infections and/or people with antibiotic resistant bacterial infections.

Accordingly, the composition of the present invention may be used to prepare a pharmaceutical drug that is beneficial for the treatment or prevention of growth of C. acnes. In an embodiment of the present invention the composition may be used curatively or prophylactically, for example, in combination with a probiotic and/or a prebiotic composition.

The combination of the composition according to the present invention and a probiotic strain provides a combined composition capable of inhibiting the growth of C. acnes in co-culture by reducing the growth of C. acnes with at least 50% as compared to the growth without the bacterial strain. Wherein growth is measured as colony forming units of C. acnes.

In an embodiment of the present invention the bacterial strain as defined herein may be the only bacteria present in the composition. The composition only comprising bacterial strains as defined herein show reduction in the growth of C. acnes with at least 50% as compared to the growth without the bacterial strain.

In a further embodiment of the present invention the one or more bacterial strain as defined herein constitute at least 10% (w/w) of the total bacterial content of the composition; such as at least 25% (w/w); e.g. at least 50% (w/w); such as at least 75% (w/w); e.g. at least 85% (w/w); such as at least 90% (w/w); e.g. at least 95% (w/w); such as at least 98% (w/w); e.g. at least 100% (w/w).

A “decrease” in growth may be “statistically significant” as compared to the growth period in the absence of the bacterial strains of the present invention, and may include a 1 percent, 2 percent, 3 percent, 4 percent, 5 percent, 6 percent, 7 percent, 8 percent, 9 percent, 10 percent, 11 percent, 12 percent, 13 percent, 14 percent, 15 percent, 16 percent, 17 percent, 18 percent, 19 percent, 20 percent, 25 percent, 30 percent, 35 percent, 40 percent, 45 percent, 50 percent, 55 percent, 60 percent, 65 percent, 70 percent, 75 percent, 80 percent, 85 percent, 90 percent, 95 percent, or 100 percent decrease.

In an embodiment of the present invention the growth inhibition may be determined as a decrease in growth of at least 25 percent. Preferably the growth inhibition is determined as a decrease in growth of at least 50 percent. Even more preferably the growth inhibition is determined as a decrease in growth of at least 90 percent.

A “decrease” in the number of microorganisms may be “statistically significant” as compared to the number of CFU/ml in the absence of the bacterial strains of the present invention, and may include a 10 percent, 15 percent, 20 percent, 25 percent, 30 percent, 35 percent, 40 percent, 45 percent, 50 percent, 55 percent, 60 percent, 65 percent, 70 percent, 75 percent, 80 percent, 85 percent, 90 percent, 95 percent, 97 percent, 99 percent, 99.9 percent or 100 percent decrease

The number of microorganisms is measured as Colony Forming Units CFU/ml.

The microorganisms according to the present invention may preferably be in isolated or purified form, where the term “isolated” means in particular that the lactic acid bacteria are derived from their culture medium including their natural medium, for example. The term “purified” is not restricted to absolute purity.

In an embodiment of the present invention the probiotic strain may be used as a live isolated microorganism in a stabilized form. Suitable methods for stabilization are known to those skilled in the art and includes freeze drying or lyophilization involving different cryoprotectants.

In a further embodiment of the present invention the strain may be used as a live isolated strain.

Preferably, the strain may be used as a live isolated stabilized strain. Even more preferably, the strain may be used as a live isolated strain stabilized by lyophilization. Even more preferably, the strain may be used as a live isolated strain stabilized by lyophilization and comprising a cryoprotectant.

The present invention relates to bacterial strains that are viable and/or are dead (killed), both forms may be included within the scope of the present invention.

Suitable methods for killing (e.g., biological, chemical or physical killing methods) are sufficiently familiar to those skilled in the art. In the present case, however, the bacterial strains may also be used in lyophilized form. The killed forms of the microorganisms can include the fermentation broth and any present metabolites.

The terms “killed” or “dead” relates to inactivated lactic acid bacteria incapable of cell division and without any metabolic activity. Dead or killed lactic acid bacteria may have intact or ruptured cell membranes.

“Lysates”, “derivatives”, “analogues”, “fractions” or “extracts” may be obtained from dead or killed lactic acid bacteria. These lysates, fractions, derivative, analogues, and extracts preferably have the properties of decreasing the transfer of a pathogenic microorganism between a surface of a first subject and a surface of a second subject, where “lysate” as well as the term “extract” refers in particular to a solution or suspension in an aqueous medium of the cells of the microorganism according to the invention and comprises, for example, macromolecules such as DNA, RNA, proteins, peptides, lipids, carbohydrates, etc. as well as cell detritus. The lysate preferably includes the cell wall or cell wall constituents including binding receptors. Methods of producing lysates are sufficiently well known to those skilled in the art and includes, for example, the use of a “French press” or enzymatic lysis, a ball mill with glass beads or iron beads. Cells can be broken open by enzymatic, physical or chemical methods. Examples of enzymatic cell lysis may include individual enzymes as well as enzyme cocktails, for example, proteases, proteinase K, lipases, glycosidases; chemical lysis may be induced by ionophores, detergents such as SDS, acids or bases; physical methods may also be implemented by using high pressures such as the French press, osmolarities, temperatures or alternating between heat and cold. Furthermore chemical, physical and enzymatic methods may of course be combined.

In a preferred embodiment the composition and/or the bacterial strains according to the present invention is suitable for treating; alleviating, suppressing; prophylaxis a disease associated with a pathogenic microorganism infection in a mammal.

The present invention also relates to a method to decrease number of C. acnes on the skin of a patient with acnes infection.

In an embodiment of the invention, the composition comprising at least one bacterial strain may be used to control the number of C. acnes on the skin of patients with inflammatory skin diseases at a level where the C. acnes does not result in an infection of the skin.

In yet an embodiment of the invention, the composition comprising at least one bacterial strain may be used to control the number of C. acnes on the skin of patients with inflammatory skin diseases wherein the level of C. acnes is below approximately 10⁷ CFU/cm³ of the skin.

It will be clear to those skilled in the art that here, as well as in all the statements of range given in the present invention, characterized by such terms as “about” or “approximately,” that the precise numerical range need not be indicated with expressions such as “about” or “approx.” or “approximately,” but instead even minor deviations up or down with regard to the number indicated are still within the scope of the present invention. In an embodiment of the present invention, the minor deviation may include a 5% deviation or less, such as a 4% deviation or less, e.g. a 3% deviation or less, such as a 2% deviation or less, e.g. a 1% deviation or less.

In an embodiment of the present invention, a biologically pure culture of one or more of the bacterial strain(s) of the present invention may be provided.

The term “effective amount” may depends upon the context in which it is being applied. In the context of administering a composition to reduce the risk of C. acnes infection, and/or administering a composition to reduce the severity of C. acnes infection and/or decreasing the amount of C. acnes in a subject, an effective amount of a composition described herein is an amount sufficient to treat and/or ameliorate an C. acnes infection, as well as decrease the severity and/or reduce the likelihood of a C. acnes infection. The decrease in the amount of pathogenic C. acnes in a subject, may be a 10 percent decrease, 20 percent decrease, 30 percent decrease, 40 percent decrease, 50 percent decrease, 60 percent decrease, 70 percent decrease, 80 percent decrease, 90 percent decrease, 95 percent decrease, 98 percent decrease, 99 percent decrease or 99.9 percent decrease in severity of C. acnes infection, or likelihood of becoming infected.

An effective amount may be administered as a composition in one or more administrations.

The effective amount of the composition can be administered as a topical administration, an oral administration or a combination thereof. Preferably as a topical administration.

In an embodiment of the present invention the composition comprising the at least one bacterial strain according to the present invention and a prebiotic.

“Prebiotics” are non-digestible food components that increase the growth of specific microorganisms. “Synbiotics” are compositions comprising at least one probiotic and at least one prebiotic. Such compositions are understood to encourage the growth of beneficial bacteria (e.g. the probiotic). Thus, powerful synbiotics are based on a combination of specific strains of probiotic bacteria with carefully selected prebiotics. They can lead to an important health benefit to a mammal.

According to another aspect of the present invention there is provided a probiotic composition comprising the probiotic microorganism and at least one more active ingredient.

Prebiotics refer to chemical products that induce the growth and/or activity of commensal microorganisms (e.g., bacteria and fungi) that contribute to the well-being of their host. Prebiotics are nondigestible carbohydrates that pass undigested through the upper part of the gastrointestinal tract and stimulate the growth and/or activity of advantageous bacteria that colonize the large bowel or skin microorganisms.

Some oligosaccharides that are used as prebiotics are fructooligosaccharides (FOS), xylooligosaccharides (XOS), polydextrose, pectins, galactooligosaccharides (GOS) or human milk oligo saccharides (HMO). Moreover, disaccharides like lactulose or some monosaccharides such as lactose or tagatose can also be used as prebiotics.

In an embodiment of the present invention at least one prebiotic compound may be comprised in the composition of the invention. In a very broad concept, prebiotics are all those compounds which can be metabolized by probiotics.

Preferably prebiotics are non-digestible or poorly digestible by a mammal. It is believed that the prebiotics, many of which are non-digestible carbohydrates, promote the growth of probiotics. Prebiotics are naturally found for example in cabbage, onions, whole grains, bananas, garlic, honey, leeks, artichokes, fortified foods and beverages, as well as dietary supplements. Prebiotics are well known in the art and when used in the present invention there is no particular limitation of the prebiotic as such.

In an embodiment however the at least one prebiotic product in the composition is selected from the following compounds and compositions: non-digestible carbohydrates, beta-glucans, mannan-oligosaccharides, inulin, oligofructose, human milk oligosaccharides (HMO), galactooligosaccharides (GOS), lactulose, lactosucrose, galactotriose, fructo-oligosaccaride (FOS), cellobiose, cellodextrins, cylodextrins, maltitol, lactitol, glycosilsucrose, betaine, Vitamin E or a variant thereof (wherein the variants are selected from alfa, beta, gamma, delta tocoferols, tocotrienols and tocomonoenols). Optionally, mannan-oligosaccharides and/or inulin may be preferred. HMOs may include lacto-N-tetraose, lacto-N-fucopentaose, lacto-N-triose, 3 “-sialyllactose, lacto-N-neofucopentaose, sialic acid, L-fucose, 2-fucosyllactose, 6”-sialyllactose, lacto-N-neotetraose and 3-fucosyllactose.

Prebiotics may also be used in topical compositions of the invention.

In an embodiment at least one of the following prebiotic compounds are used in the topical composition of the invention; lactose, beta-glucans, mannan-oligosaccharides, inulin, oligofructose, galactooligosaccharides (GOS), lactulose, lactosucrose, galactotriose, fructo-oligosaccaride (FOS), cellobiose, cellodextrins, cylodextrins, maltitol, lactitol, glycosilsucrose, betaine, Vitamin E or a variant thereof (wherein the variants are selected from alfa, beta, gamma, delta tocoferols, tocotrienols and tocomonoenols), lacto-N-tetraose, lacto-N-fucopentaose, lacto-N-triose, 3 ′-sialyllactose, lacto-N-neofucopentaose, sialic acid, 2-fucosyllactose, 6′-sialyllactose, lacto-N-neotetraose and 3-fucosyllactose. Optionally, lactose and/or mannan-oligosaccharides and/or inulin may be preferred.

D- and L-fucose strengthen natural defense of skin, stimulate epidermis immune defense and/or prevent and/or treat cutaneous autoimmune disease. In an embodiment of the invention the composition comprises D- or L-fucose.

In an embodiment of the invention the composition further comprises L-fucose in a concentration in the composition of 10 mM to 500 mM.

According to still further features in the described preferred embodiments the composition further comprises at least one active ingredient.

In an embodiment of the present invention the composition comprising at least on bacterial strain of the invention in combination with at least one further probiotic microorganism selected from the group consisting of another bacteria, a yeast or a mold.

The composition according to the present invention may comprise at least one bacterial strain according to the present invention in combination with at least one further probiotic microorganism, wherein the at least one further probiotic microorganism may be selected from but not restricted to: Bifidobacterium lactis DSM10140, B. lactis LKM512, B. lactis DSM 20451, Bifidobacterium bifidum BB-225, Bifidobacterium adolescentis BB-102, Bifidobacterium breve BB-308, Bifidobacterium longum BB-536 from Zaidanhojin Nihon Bifizusukin Senta (Japan Bifidus Bacteria Center), Bifidobacterium NCIMB 41675 described in EP2823822. Bifidobacterium bifidum BB-225, Bifidobacterium adolescentis BB-102, Bifidobacterium breve BB-308, Bifidobacterium lactis HNO19 (Howaru) available from DuPont Nutrition Biosciences ApS, Bifidobacterium lactis DN 173 010 available from Groupe Danone, Bifidobacterium lactis Bb-12 available from Chr. Hansen A/S, Bifidobacterium lactis 420 available from DuPont Nutrition Biosciences ApS, Bifidobacterium breve Bb-03, B. lactis BI-04, B. lactis Bi-07 available from DuPont Nutrition Biosciences ApS, Bifidobacterium bifidum Bb-02, Bifidobacterium bifidum Bb-06, Bifidobacterium longum KC-1 and Bifidobacterium longum 913 (DuPont Nutrition Biosciences ApS), Bifidobacterium breve M-16V (Morinaga) and/or a Lactobacillus having a probiotic effect and may be any of the following strains; Lactobacillus rhamnosus LGG (Chr. Hansen), Lactobacillus acidophilus NCFM (DuPont Nutrition Biosciences ApS), Lactobacillus bulgaricus 1260 (DuPont Nutrition Biosciences ApS), Lactobacillus paracasei Lpc-37 (DuPont Nutrition Biosciences ApS), Lactobacillus rhamnosus HN001 (Howaru) available from DuPont Nutrition Biosciences ApS, Streptococcus thermophilus 715 and Streptococcus thermophilus ST21 available from DuPont Nutrition Biosciences ApS, Lactobacillus paracasei subsp. paracasei CRL431 (ATCC 55544), Lactobacillus paracasei strain F-19 from Medipharm, Inc. L. paracasei LAFTI L26 (DSM Food Specialties, the Netherlands) and L. paracasei CRL 431 (Chr. Hansen), Lactobacillus acidophilus PTA-4797, L. salivarius Ls-33 and L. curvatus 853 (DuPont Nutrition Biosciences ApS). Lactobacillus casei ssp. rhamnosus LC705 is described in Fl Patent 92498, Valio Oy, Lactobacillus DSM15527 (Bifodan), Lactobacillus DSM15526 (Bifodan), Lactobacillus rhamnosus GG (LGG) (ATCC 53103) is described in U.S. Pat. No. 5,032,399 and Lactobacillus rhamnosus LC705 (DSM 7061), Propionic acid bacterium eg. Propionibacterium freudenreichii ssp. shermanii PJS (DSM 7067) described in greater details in FI Patent 92498, Valio Oy, Nitrosomonas eutropha D23 (ABlome), Staphylococcus hominis strains A9, C2, AMT2, AMT3, AMT4-C2, AMT4-GI, and/or AMT4-D12. (all from Matrisys Bioscience), Staphylococcus epidermidis strains M034, M038, All, AMT1, AMT5-05, and/or AMT5-G6 (all from Matrisys Bioscience), L. plantarum YUN-V2.0 (BCCM LMG P-29456), L. pentosus YUN-V1.0 (BCCN LMG P-29455), L. rhamnosus YUN-S1.0 (BCCM LMG P-2961), Weissella viridescens LB10G (DSM 32906), Lactobacillus paracasei LB113R (DSM 32907), Lactobacillus plantarum LB244R (DSM 32996), Lactobacillus paracasei LB116R (DSM 32908), Enterococcus faecium LB276R (DSM 32997), Lactobacillus plantarum LB316R (DSM 33091), Lactobacillus plantarum LB356R (DSM 33094), Lactobacillus plantarum LB312R (DSM 33098); and/or any combinations hereof.

In an embodiment of the present invention the composition comprises at least one bacterial strain as defined herein in combination with at least one strain selected from the group of lactic acid bacteria being able to improve tight junction integrity.

In a further embodiment of the present invention the composition comprises at least one bacterial strain as defined herein in combination with at least one additional strain selected from the group of Lactobacillus rhamnosus LGG (Chr. Hansen), Lactobacillus acidophilus NCFM (DuPont), Lactobacillus salivarius Ls-33 (DuPont), Propionibacterium jensenii P63 (DuPont), Bifidobacterium lactis 420 (DuPont) and L. acidophilus La-14 (DuPont); and/or the cell lysate and/or the soluble metabolite of the probiotic strain.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of disease, stabilized (i.e., not worsening) state of disease, prevention of disease, delay or slowing of disease progression, and/or amelioration or palliation of the disease state.

The decrease can be a 10 percent, 20 percent, 30 percent, 40 percent, 50 percent, 60 percent, 70 percent, 80 percent, 90 percent, 95 percent, 98 percent or 99 percent decrease in severity of complications or symptoms.

In an embodiment of the present invention a method of treating the skin of mammals may be provided. The method comprising administering to a subject (e.g. a mammal) in need thereof a therapeutically effective amount of at least one bacterial strain, thereby treating skin to reduce colonization and/or carrier level and/or infection.

In a further embodiment of the present invention the skin colonization may be caused by an antibiotic resistant microorganism. The skin colonization may be caused by resistant C. acnes.

The present invention may provide several advantages. In particular, insofar as there is a detrimental effect to the use of anti-acne products because of the potential to produce resistant microbial species, it is desirable to have an antimicrobial treatment which does not utilize conventional antimicrobial agents. Hence, the present invention does not contribute to the production of future generations of antibiotic resistant pathogens.

The present invention may provide several advantages. In particular, insofar as there is a detrimental effect to the use of anti-acne products because these results in significant dryness of the skin, it is desirable to have an antimicrobial treatment which does not cause any dryness of the skin. Hence, the present invention does not result in dry skin.

The present invention may provide several advantages. In particular, insofar as there is a detrimental effect to the use of anti-acne products because of the potential to cause irritation and/or inflammation of the skin, it is desirable to have an antimicrobial treatment which does not cause irritation or further inflammation of the skin.

The present invention may provide several advantages. In particular, insofar as there is a detrimental effect to the use of anti-acne products because of the resulting discoloration (pigmentation) of skin and sun sensitivity, it is desirable to have an antimicrobial treatment which does not cause any discoloration or sun sensitivity of the skin. Hence, the present invention does not result in either coloration or sun sensitivity. A further advantage of the present invention is that the present invention decreases the appearance of pigmentation and discoloration as a further benefit to the antimicrobial activity against C. acnes.

A preferred embodiment of the present invention relates to a pharmaceutical composition comprising the composition according to the present invention for use as a medicament.

Moreover, a preferred embodiment of the present invention relates to a pharmaceutical composition comprising the composition according to the present invention for use in treating; alleviating, suppressing; prophylaxis; and/or preventing growth of a pathogenic micro-organism.

Furthermore, a preferred embodiment of the present invention relates to a pharmaceutical composition comprising the composition according to the present invention, comprising the one or more bacterial species capable of co-aggregating one or more C. acnes strains for use in treating; alleviating, suppressing; prophylaxis; and/or preventing of acne vulgaris.

Yet an embodiment of the present invention relates to A method of treating; alleviating, suppressing; prophylaxis; and/or preventing growth of a pathogenic micro-organism, in particular one or more pathogenic C. acnes strains, e.g. acne vulgaris in a mammal such as a human, the method comprising administering to a mammal in need thereof a therapeutically effective amount of a composition as defined herein comprising one or more bacterial species, e.g. one or more lactic acid bacteria as defined herein, such as one or more bacterial strain(s) selected from the group consisting of:
LB356R (Lactobacillus plantarum LB356R, deposited under DSM 33094)
LB244R (Lactobacillus plantarum LB244R, deposited under DSM 32996)
LB349R (Leuconostoc mesenteriodes LB349R, deposited under DSM 33093)
LB10G (Weissella viridescens LB10G, deposited under DSM 32906).

Thus, one aspect of the invention relates to new isolated bacterial strains and/or a composition comprising one or more bacterial strain, with antimicrobial activity against pathogenic C. acnes.

Another aspect of the invention relates to new isolated bacterial strains with growth inhibitory activity against pathogenic C. acnes.

Another aspect of the invention relates to new isolated bacterial strains which are able to co-aggregate with pathogenic C. acnes.

Another aspect of the present invention relates to compositions comprising bacterial strains with antimicrobial activity and/or growth inhibitory activity and/or co-aggregative activity against pathogenic C. acnes.

Yet another aspect of the present invention is to provide use of a composition comprising bacterial strains with antimicrobial activity and/or growth inhibitory activity and/or co-aggregative activity against pathogenic C. acnes.

Still another aspect of the present invention is to provide a composition for use to treat infection caused by C. acnes.

Deposit of Biological Material

The following biological material, microorganisms, have been deposited at the with the German Collection for Microorganisms and Cell Cultures as
Lactobacillus plantarum LB356R, deposited under DSM 33094
Lactobacillus plantarum LB244R, deposited under DSM 32996
Leuconostoc mesenteriodes LB349R, deposited under DSM 33093
Weissella viridescens LB10G, deposited under DSM 32906

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1 Strain Screening Identification

Samples

For identification and selection of bacterial strain(s) according to the invention, a strain collection of lactic acid bacteria (LAB) was established. Samples from different origins, such as homemade sauerkraut, kimchi and healthy human donor samples (vaginal, oral, anal, skin) were collected for isolation of at least 995 lactic acid bacteria. The samples were collected on Man Rogosa Sharp (MRS, Sigma-Aldrich) broth and agar cultured anaerobically at 37° C. overnight or until colony formation. The isolates are plated and sub-cultured until pure colonies were obtained. The pure colonies are stored in MRS broth with 25% glycerol at −80° C. for future use. Strains were identified using 16S rRNA Sanger sequencing standard methods.

### Example 2—Co-Aggregation

C. acnes strains were obtained from BEI Resources were used as research test organisms for the screen.

| | | Phylotype | |
|---|---|---|---|
| Strain | ATCC ref | (recA) | Ribotype |
| Cutibacterium acnes | HM-554 | II | Ribotype RT6 - non- |
| HL110PA3 | | | pathogenic |
| Cutibacterium acnes | HM-555 | II | Ribotype RT6 - non- |
| HL110PA4 | | | pathogenic |
| Cutibacterium acnes | HM-552 | IB | Ribotype RT8 - acne |
| HL110PA1 | | | |
| Cutibacterium acnes | HM-523 | IB | Ribotype RT8 - acne |
| HL053PA2 | | | |
| Cutibacterium acnes | HM-513 | IA | Ribotype RT5 - acne |
| HL043PA1 | | | |
| Cutibacterium acnes | HM-512 | IA | Ribotype RT4 - acne |
| HL038PA1 | | | |

C. acnes ATCC 6919 (America Type Culture Collection) was used a typestrain for C. acnes.

Lactobacillus rhamnosus LGG (Chr. Hansen) was used as a control probiotic strain in all experiments, and YUN product ACN+cream (YUN Probiotherapy, webshop: www.shop.yun.be) comprising live lactic acid bacteria was used as a control of commercial available probiotic lactic acid bacteria for topical use. The L. rhamnosus LBB and YUN strains were grown as LAB on MRS as described in the examples.

Co-aggregation was determined according to known methods Cisar, J. O. et al. (1979). “Specificity of Coaggregation Reactions between Human Oral Streptococci and Strains of Actinomyces Viscosus or Actinomyces Naeslundii.” Infection and Immunity 24 (3): 742-52. Inoculum of all Lactic acid bacteria (LAB) were grown in MRS broth anaerobically overnight and the C. acnes strains were grown in BHI broth anaerobically at 37° C. to a cell density of approximately 10⁸ CFU/ml (2-3 days). Overnight cell samples were harvest by centrifugation (6000 rpm for 2 min), and supernatants were removed from the pellet. The pellets were washed twice in 1×PBS buffer.

Cell pellet was resuspended in 1×PBS and 500 μl of C. acnes and LAB strains were aliquoted into 24 well plates. The plates were incubated on a shaker (200 rpm). Auto- and co-aggregation formation was observed visually after 1 hour, 2 hours, 3 hours and 24 hours.

As a control for self-aggregation (auto-aggregation), each strain was mixed with PBS in a final volume of 1500 ul in each well.

Co-aggregation was determined by mixing C. acnes and LAB strains. Each strain suspended in PBS buffer, and mixed 1:1 in a final volume of 1500 ul in each well.

The plates were incubated on a shaker at approximately 200 rpm for 24 hours. Co-aggregation formation is observed after 1 hour, 2 hours, 3 hours and 24 hours. The formation of co-aggregation was scored visually from 1-5 using the following scale:
1: No aggregation
2: Visual initial aggregation
3: Formation of aggregates <0.5 mm
4: Formation of aggregates >0.5 mm and <1 mm
5: Formation of aggregates >1 mm

TABLE 1
Co-aggregation measured using a visual evaluation from 1 to 5 (see scale above).
Data shown for 1 hour and 24 hours incubation

| | HM-552 | | HM-523 | | HM-513 | | HM-512 | |
|---|---|---|---|---|---|---|---|---|
| LAB | 1 h | 24 h | 1 h | 24 h | 1 h | 24 h | l h | 24 h |
| LB10G | 2 | 3 | 2 | 3 | 2 | 3 | 3 | 4 |
| LB244R | 4 | 4 | 3 | 3 | 3 | 4 | 4 | 4 |
| LB349R | 3 | 3 | 3 | 3 | 2 | 3 | 3 | 4 |
| LB356R | 4 | 5 | 4 | 5 | 5 | 5 | 5 | 5 |
| LGG | 1 | 1 | 1 | 1 | 1 | 1 | 1 | 1 |
| YUN product | 1 | 1 | 1 | 1 | 1 | 1 | 1 | 1 |

| | HM-554 | | HM-555 | | ATCC6919 | |
|---|---|---|---|---|---|---|
| LAB | 1 h | 24 h | 1 h | 24 h | 1 h | 24 h |
| LB10G | 1 | 3 | 2 | 2 | 1 | 2 |
| LB244R | 2 | 2 | 2 | 2 | 2 | 3 |
| LB349R | 2 | 3 | 3 | 2 | 1 | 2 |
| LB356R | 2 | 3 | 3 | 3 | 3 | 4 |
| LGG | 1 | 1 | 1 | 1 | 1 | 1 |
| YUN product | 1 | 1 | 1 | 1 | 1 | 1 |

34 LAB strains were identified as being able to co-aggregate with pathogenic C. acnes strains, in table 1 the co-aggregation of 4 strains (LB10G, LB244R, LB349R and LB356R) are listed, these strains co-aggregate relatively more with the pathogenic ribotypes of C. acnes (RT4, RT5 and RT8) as compared to the non-pathogenic ribotype (RT6).

The ATCC type strain behave in general slightly different in the test the strain is also easier to grow and can be grown anaerobically overnight in BHI to OD(600)>1.

The controls LGG and the strains in the commercial product from YUN does not show any ability to co-aggregate with C. acnes.

Co-aggregation can inhibit growth of the organism and influence the ability to create biofilm. Thus, LAB being able to co-aggregate with C. acnes can inhibit the biofilm formation and decrease virulence of the acne strain.

### Example 3—Effect of pH on Co-Aggregation

Co-aggregation was determined according to known methods Cisar, J. O. et al. (1979) as described above.

Inoculum of all Lactic acid bacteria (LAB) were grown in MRS broth anaerobically overnight and the C. acnes strains were grown in BHI broth anaerobically at 37° C. to a cell density of approximately 10⁸ CFU/ml (2-3 days). Overnight cell samples were harvest by centrifugation (6000 rpm for 2 min), and supernatants were removed from the pellet. The pellets were washed twice in the pH adjusted buffer used for the co-aggregation test, respectively.

MOPS (3-(N-morpholino)propanesulfonic acid) obtained from Merck (product 69947) and MES (2-(N-morpholino)ethanesulfonic acid) obtained from Sigma (product M2933) buffers were used in the concentration 100 mM. MES was used for pH 4.5, pH 5, pH 5.5, pH 6 and pH 6.5. MOPS was used for pH 6.5, pH 7, pH 7.5 and pH 8. Co-aggregation was found to be slightly less in the inert buffers MES and MOPS as compared to PBS buffer. The MES and MOPS allow for evaluation of pH effect on co-aggregation in a broad spectrum of pH value limiting any influence of the buffer.

Cell pellet of each strain was resuspended in each buffer and 500 μl of C. acnes and LAB strains were aliquoted into 24 well plates for each pH. The plates were incubated on a shaker (200 rpm). Auto- and co-aggregation formation was observed visually after 1 hour, 2 hours, 3 hours and 24 hours.

As a control for self-aggregation (auto-aggregation), each strain was at each pH value was included in a final volume of 1000 ul in the well. Self-aggregation was found not to be significantly different at the different pH values. No significant self-aggregation have been observed for any of the tested strains.

Co-aggregation was determined by mixing C. acnes and LAB strains. Each strain suspended in PBS buffer, and mixed 1:1 in a final volume of 1000 ul in each well.

The plates were incubated on a shaker at approximately 200 rpm for 24 hours. Co-aggregation formation is observed after 1 hour, 2 hours, 3 hours and 24 hours. The formation of co-aggregation was scored visually from 1-5 using the following scale:
1: No aggregation
2: Visual initial aggregation
3: Formation of aggregates <0.5 mm
4: Formation of aggregates >0.5 mm and <1 mm
5: Formation of aggregates >1 mm

TABLE 2
Co-aggregation measured using a visual evaluation from 1 to
5 (see scale above). Data shown for 1 hour incubation and
for the 2 strains LB244R and LB356R, and the pathogenic C. acnes
HM-513 and the non-pathogenic C. acnes HM-554.

| | LB244R + | LB244R + | LB356R + | LB356R + |
|---|---|---|---|---|
| Buffer | HM513 | HM554 | HM513 | HM554 |
| PBS | 3 | 2 | 5 | 2 |
| MES 4.5 | 5 | 4 | 5 | 3 |
| MES 5.0 | 5 | 3 | 5 | 3 |
| MES 5.5 | 5 | 3 | 5 | 3 |
| MES 6.0 | 4 | 3 | 5 | 3 |
| MES 6.5 | 4 | 2 | 5 | 2 |
| MOPS 6.5 | 4 | 2 | 5 | 2 |
| MOPS 7.0 | 3 | 2 | 4 | 2 |
| MOPS 7.5 | 2 | 1 | 4 | 1 |
| MOPS 8.0 | 2 | 1 | 3 | 1 |

For the strains being able to co-aggregate the result was in general the same. Decrease in pH in results increased co-aggregation. The 4 strains; LB244R, LB356R, LB10G and LB349R all resulted in an increased co-aggregation when decreasing the pH. The ratio between co-aggregation with pathogenic versus non-pathogenic C. acnes was constant and appears not to be significantly affected by pH change. Thus, at low pH, the strains still co-aggregate more with the pathogenic C. acnes as compared to the non-pathogenic C. acnes.

The controls LGG and the strains in the commercial product from YUN does not show any ability to co-aggregate with C. acnes in any of the buffers

Co-aggregation can inhibit growth of the organism and influence the ability to create biofilm. Thus, LAB being able to co-aggregate with C. acnes can inhibit the biofilm formation and decrease virulence of the acne strain. This effect is therefore expected to be further improved at pH values below 7.

### Example 4—Spot on Lawn Assay

Spot on lawn test for growth inhibition and antimicrobial metabolites were tested using the methods described in Zhang P. et al. (2015) Interstrain interactions between bacteria isolated from vacuum-packaged refrigerated beef. Appl Environ Microbiol 81:2753-2761. doi:10.1128/AEM.03933-14 and Arena, M. P. et al. (2016) Use of Lactobacillus plantarum Strains as a Bio-Control Strategy against Food-Borne Pathogenic Microorganisms. Frontiers in Microbiology 7 (APR): 1-10. https://doi.org/10.3389/fmicb.2016.00464.

Bacterial strain isolates from example 1 were cultured from storage samples into 2 mL of MRS broth in 24-well plates. Each C. acnes test strains were cultured anaerobically in approximately 200 mL of BHI broth in Erlenmeyer flask for 48-72 hours (OD600>1). The LAB isolates were grown overnight at 37° C. The cell density of the overnight culture of C. acnes is adjusted in BHI broth to an optical density at 600 nm (OD600) of 1 and hereafter diluted in PBS buffer to a 10⁻² dilution. Two hundred microliters of cell suspension were spread onto BHI agar plates. The plates with C. acnes lawn were left for drying for appr. 10-20 minutes in sterile air. Three replicates of 20 μL of isolated LAB was spotted onto the C. acnes lawn. The plates were left for drying and hereafter incubated at 37° C. anaerobically for 48 hours. The inhibition zone is measured in mm as the clearing zone around the spot, and diameter is measured as the full diameter of the spot. Growth inhibition is observed as the LAB being able to overgrow the C. acnes strain in the spot area indicated in table 2 as a (+). If the C. acnes strain is able to outgrow the spotted bacterial strain, then there is no detected grow inhibition indicated in table 2 as a (−).

15 strains were able to out-grow all C. acnes strains, 5 LAB strains were identified as having a significant growth inhibitory effect on C. acnes and also an inhibition zone of more than 1 mm around spot.

L. rhamnosus LGG (Chr. Hansen) and Yun product were used as control commercial probiotic strains. Neither L. rhamnosus nor YUN strains were able to growth inhibit any of the pathogenic C. acnes test strains nor did L. rhamnosus LGG or YUN product give any clearing zone.

C. acnes ATCC 6919 appears to be easier to growth inhibit and numerous strains were identified to have growth inhibitory activity against ATCC 6919 but appears not to have any growth inhibitory activity against the pathogenic isolates (RT4, RT5 and RT8) or even against the non-pathogenic isolates (RT6),

TABLE 3
Spot assay with type strain (C. acnes ATCC 6919) as target,
inhibitor zone measured per strain with an effect.
Type strain C. acnes

| Strains | *Diameter [mm] | **The inhibitor zone [mm] |
|---|---|---|
| LB10G | 15 | 3.1 |
| LB244R | 16 | 2.8 |
| LB349R | 17 | 3.7 |
| LB356R | 12 | 1.5 |

*The diameter was an average of three measurements of the inhibitory zone.
**The length of the inhibitor zones was a average of three measurements from the periphery of bacteria spot to growth of C. acnes.

TABLE 4
Spot assay with HM-523 as target strain,
inhibitor zone measured per strain with an effect.
HM-523

| Strains | *Diameter [mm] | **The inhibitor zone [mm] |
|---|---|---|
| LB10G | 14 | 2 |
| LB244R | 14 | 2 |
| LB349R | 14 | 2 |
| LB356R | 13 | 1 |

*The diameter was an average of three measurements of the inhibitory zone.
**The length of the inhibitor zones was an average of three measurements from the periphery of bacteria spot to growth of HM-523.

TABLE 5
Growth inhibition and antimicrobial metabolites. Inhibition zone is determined as
average of three measurements.

| | | | | | Inhibition zone: clearing around | | | |
|---|---|---|---|---|---|---|---|---|
| | Growth inhibition in spot. | | | | the spot (mm) | | | |
| | HM- | HM- | HM- | HM- | HM- | HM- | HM- | HM- |
| LAB | 523 | 513 | 512 | 555 | 523 | 513 | 512 | 555 |
| LB10G | + | + | + | + | 2 | 3 | 2 | 2 |
| LB244R | + | + | + | + | 2 | 3 | 3 | 2 |
| LB349R | + | + | + | + | 3 | 3 | 3 | 3 |
| LB356R | + | + | + | + | 1.5 | 1.7 | 1.5 | 2 |
| LGG | − | − | − | − | 0 | 0 | 0 | 0 |

### Example 5—Evaluation of Bacterial Lysates

Bacterial lysates were produced using 3 different methods.

LAB isolate (LB356R) were grown in MRS overnight at 37° C.

Lysate 1: Tube with 15 ml of overnight culture were placed in an ice-batch and cell were lysed by sonication for 30 min using a Q125 sonicator (QSonica).

Lysate 2: 15 ml of overnight culture were adjusted with 0.1M HCl to pH 3 incubated for 2 days at 45 degrees Celsius.

Lysate 3: 15 ml of overnight culture were added 5% SDS (Sigma-Aldrich L3771) and stored at −20 degrees Celsius for 1 hour, incubated at 50 degrees Celsius for 1 hour. Cycles changing temperature from −20 to +50 degrees were done for 2×8 hours Each lysate was evaluated for viable cells after the lysate processes, respectively.

Lysates were evaluated for maintained activity against C. acnes HM-512 by testing for ability to co-aggregate as described in example 3, and also for growth inhibitory activity by co-incubating each lysate with BHI inoculated with HM-512 approximately to 10⁴ CFU/ml and follow growth and growth inhibition by measuring OD (600 nm) using a spectrophotometer.

All three lysates were shown to growth inhibit HM-512. Lysate 1 and 2 were found to co-aggregate comparable to viable LB356R cells, whereas the lysate 3 were found to co-aggregate less than the viable cells with a co-aggregation score of 3 using the scale in example 2.

### Example 6

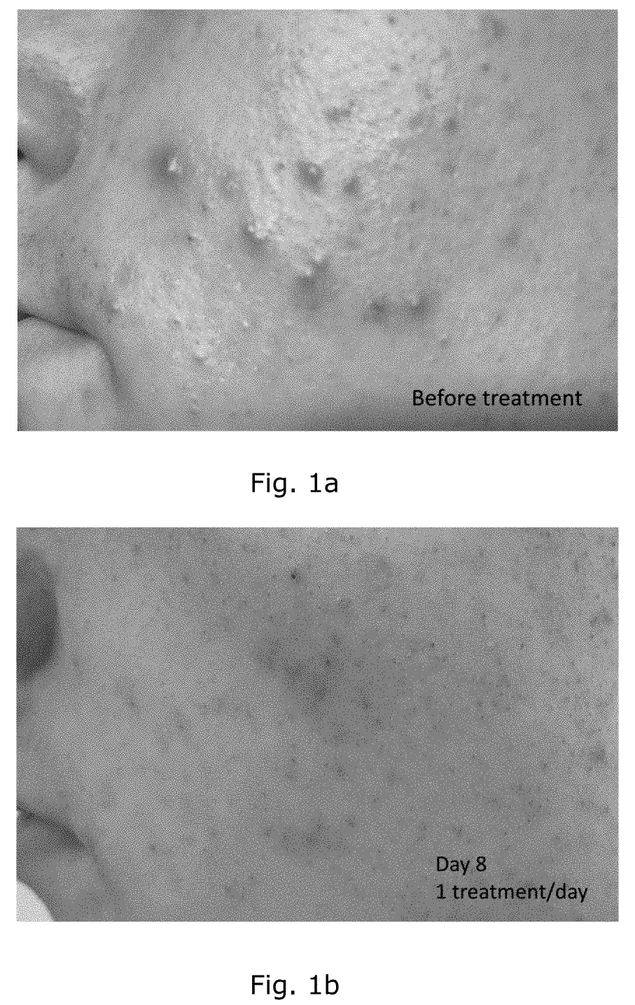

Strain LAB strain LB356R was formulated in vegetable oil comprising jojoba oil and almond oil (1:1). The strain was added in the concentration of approximately 10⁸ CFU/ml oil. 10 teenagers (age 15-19) with acne-prone skin were treated with the composition once a day. Visual evaluation of acne severity was determined by pictures before treatment (FIG. 1a) and after treatment (FIG. 1b). Visual improvement was observed for all test persons and as average the improvement was detectable after 3-4 days treatment.

### Example 7

Anti-inflammatory activity of the LAB cells were determined on 3 D tissue model of Reconstructed Human Epidermidis (RHE) manufactured by Episkin (Lyon, France)(Batch 19-RHE-164). The RHE cells were treated with each LAB strain. Overnight cultures of each strain in MRS was diluted in saline to OD=0.8 and incubated with the RHE cells for 4 hours. Gene expression was determined by qRT-PCR of TSLP, TNF-α, IL-8, TLR-2 and HBD2. RNA integrity was evaluated by loading the extracted RNA on agarose gel 1%: ribosomial band 18S and 28s were detected. The High Capacity cDNA Reverse Transcription kit was used to synthetize cDNA from RNA. For the real time PCR, Applied Biosystems 7500 Fast Real Time PCR with fluorescent based PCR Taqman assays were used. GAPDH was used as an endogenous control gene to normalize input amounts. A significant HBD2 up-regulation was observed for all 4 LAB strains. the probiotics strains were effective in boosting the host innate immune defences against infection.

### Example 8

Biofilm formation was determined as an ability of a microorganism to initiate binding to a surface and growing on the surface.

Inoculum of all Lactic acid bacteria (LAB) were grown in MRS broth anaerobically overnight and the C. acnes strains were grown in BHI broth anaerobically at 37° C. to a cell density of approximately 10⁸ CFU/ml (2-3 days). Overnight cell samples were harvest by centrifugation (6000 rpm for 2 min), and supernatants were removed from the pellet. The pellets were washed twice in 1×PBS buffer.

Cell pellet was resuspended in BHI broth and diluted to a cell concentration of approximately 10⁶ CFU/ml. 100 μL of C. acnes and 100 μL LAB strain were aliquoted into 96 well plates (Sigma Cell culture plate SIAL0596-50EA, flat bottom). The plates were incubated 1 hour at 37° C. to allow the cells to attach to the bottom surface of the well. The microtiter plates were emptied from broth and the bottom of the well washed once with PBS.

200 μL sterile BHI broth was added to each well and level of attached cells to the surface was determined by absorbance measurements using a standard curve for each strain with the correlation between OD measurement (detection at 600 nm) and cell number determined by plate counting as CFU/ml and calculated to the number of attached viable CFU/well.

Initial early biofilm formation was followed by microscopy and image analysis using the oCelluScope from BioSense solutions, Denmark. Biofilm outgrowth was followed for 4 hours at 37° C. (anaerobically). Biofilm outgrowth was found to correlate directly with the number of surface attached cells.

Controls were included with selective medium (MRS) for LAB to reduce the contribution of LAB to the biofilm. As well as a control for single culture biofilm formation of each acne strain. The oCelluScope allow for a visual and image analysis to evaluate the dominating strain in the biofilm based on cell morphology.

TABLE 6
Initial attachment to surface (Cell number attached to bottom
of well, number is shown as average of triplet measurement):

| | HM-523 | HM-513 | HM-512 | HM-554 | HM-555 |
|---|---|---|---|---|---|
| No LAB | 6.4 × 10³ | 3.5 × 10³ | 8.3 × 10³ | 2 × 10² | 4 × 10¹ |
| LB10G | 3.1 × 10¹ | 3.1 × 10¹ | 3.1 × 10¹ | 3.1 × 10¹ | 3.1 × 10¹ |
| LB244R | 1.4 × 10¹ | 1.4 × 10¹ | 1.4 × 10¹ | 1.4 × 10¹ | 1.4 × 10¹ |
| LB349R | 0.6 × 10¹ | 0.6 × 10¹ | 0.6 × 10¹ | 0.6 × 10¹ | 0.6 × 10¹ |
| LB356R | 0.1 × 10¹ | 0.1 × 10¹ | 0.1 × 10¹ | 0.1 × 10¹ | 0.1 × 10¹ |
| LGG | 2.8 × 10³ | 3.4 × 10³ | 4.1 × 10³ | 3.9 × 10¹ | 7.4 × 10¹ |
| YUN | 4.7 × 10³ | 2.1 × 10³ | 3.4 × 10³ | 1.3 × 10³ | 4.8 × 10¹ |
| product | | | | | |

It was observed that non-pathogenic strains (HM-554 and HM-555) in general attach less to surfaces than the pathogenic strains (HM-523, HM-513 and HM-512). Level of initial attachment for non-pathogenic strains when no probiotic strain was present was approximately 10 to 500 Cells per well. Whereas the initial attachment for pathogenic strains was approximately 1000 to 10000 cells per well when no probiotic strains were present. The presence of LB10G, LB244R, LB349R and LB356R significantly inhibited initial attachment of pathogenic strains. A 2 log unit reduction in initial attachment was thus observed for these 4 probiotic strains, this reduction was the only significant change measured. None of the two control LAB strains (LGG and YUN) was observed to have any effect on preventing initial attachment to surfaces.

### REFERENCES

Fitz-Gibbon et al. 2013, J. Invest. Dermatol. 133:2152-60. doi:10.1038/jid.2013.21
McDowell et al. 2013, PLoS ONE 8(9): e70897
Dreno et al. 2018, JEADV 32 (suppl. 2), 5-14. doi:10.1111/jdv.15043
WO 2017/220525

1. A composition comprising one or more of bacterial strains wherein the one or more of bacterial strains exhibit a stronger inhibition of pathogenic Cutibacterium acnes strains compared to non-pathogenic Cutibacterium acnes strains.

2. The composition according to claim 1, wherein the inhibition is a growth inhibition, co-aggregation inhibition, biofilm inhibition, or a combination hereof, of the C. acnes strains.

3. The composition according to claim 1, wherein the composition comprising one or more of bacterial strains capable of co-aggregating stronger to one or more pathogenic C. acnes strains relative to the capability of the one or more bacterial strains to co-aggregation to one or more non-pathogenic C. acnes strains.

4. The composition according to claim 1, wherein the inhibition of pathogenic C. acnes strains is 10% stronger or more relative to the inhibition of the non-pathogenic C. acnes strains.

5. The composition according to claim 1, wherein the one or more pathogenic C. acnes strains is selected from a Cutibacterium acnes ribotype RT4; ribotype RT5; and ribotype RT8.

6. The composition according to claim 1, wherein the one or more bacterial strains is one or more lactic acid bacterial strains, preferably, the one or more lactic acid bacterial strains is a Lactobacillus strain; a Leuconostoc strain; and/or a Weissella strain, preferably, the bacterial strains is a Lactobacillus strain.

7. The composition according to claim 6, wherein the Lactobacillus strains is selected from Lactobacillus plantarum, preferably, the Lactobacillus plantarum is selected from Lactobacillus plantarum LB356R, deposited under DSM 33094 or from Lactobacillus plantarum LB244R, deposited under DSM 32996; or a combination hereof the Leuconostoc strains is selected from Leuconostoc mesenteriodes, preferably, the Leuconostoc mesenteriodes is selected from Leuconostoc mesenteriodes LB349A, deposited under DSM 33093; and/or the Weissella strains is selected from Weissella viridescens, preferably, the Weissella viridescens is selected from Weissella viridescens LB10G, deposited under DSM 32906.

8. The composition according to claim 1, wherein the co-aggregation forms aggregates within 1 hour having an aggregate particle size above 0.1 mm.

9. The composition according to claim 1, wherein the composition is a topical composition, an oral composition, or a rectal composition.

10. The composition according to claim 1, wherein the one or more bacterial strains is provided as one or more viable strains.

11. The composition according to claim 1, wherein the concentration of the one or more bacterial strains is in the range of 10³ to 10¹⁴ colony forming units (CFU).

12. The composition according to claim 1, wherein the pH of the composition is pH 6.5 or below.

13. A pharmaceutical composition comprising the composition according to claim 1, for use as a medicament.

14. A method of treating; alleviating, suppressing; prophylaxis; and/or
preventing growth of a pathogenic micro-organism in a subject in need thereof comprising administering a therapeutically effective amount of the composition of claim 1 to the subject.

15. A method of treating; alleviating, suppressing; prophylaxis; and/or preventing of acne vulgaris in a subject in need thereof comprising administering a therapeutically effective amount of the composition of claim 1 to the subject.
